(19) United States
(12) Patent Application Publication
(10) Pub. No.: US 2022/0120741 A1
(43) Pub. Date: Apr. 21, 2022

(54) SARS-CoV2 Antigen Lateral Flow Assay Detection Device and Methods of Using the Same

(71) Applicant: Becton, Dickinson and Company, Franklin Lakes, NJ (US)

(72) Inventors: Huimiao Ren, San Diego, CA (US); Ashley Orlowski, San Diego, CA (US); Richard R. Anderson, Encinitas, CA (US)

(21) Appl. No.: 17/490,419

(22) Filed: Sep. 30, 2021

Related U.S. Application Data
(60) Provisional application No. 63/093,569, filed on Oct. 19, 2020.

(51) Int. Cl. G01N 33/543; G01N 33/569

(52) U.S. Cl. CPC G01N 33/54388; G01N 2469/10; G01N 2333/165; G01N 33/56983

(57) ABSTRACT

Lateral flow assay (LFA) devices for detecting whether SARS-CoV-2 nucleocapsid protein is present in a sample are provided. Aspects of the LFA devices include: a sample receiving region; a conjugate region downstream from the sample receiving region that includes test particulate labels made up of label particles conjugated to first and second specific binding members that specifically bind to the SARS-CoV-2 nucleocapsid protein; and a detection region downstream from the conjugate region which includes an immobilized capture specific binding member that specifically binds to the SARS-CoV-2 nucleocapsid protein. Also provided are methods of using the LFA devices, as well as readers, systems and kits for use in the same.

### CROSS-REFERENCE

Pursuant to 35 U.S.C. § 119 (e), this application claims priority to the filing date of U.S. Provisional Patent Application Ser. No. 63/093,569 filed Oct. 19, 2020; the disclosures of which applications are incorporated herein by reference in their entirety.

### INTRODUCTION

Coronaviruses are enveloped, positive-sense single-stranded RNA viruses. They have the largest genomes (26-32 kb) among known RNA viruses, and are phylogenetically divided into four genera (alpha, beta, gamma, delta), with beta-coronaviruses further subdivided into four lineages (A, B, C, D). Coronaviruses infect a wide range of avian and mammalian species, including humans. Of the six known human coronaviruses, four of them (HCoV-OC43, HCoV-229E, HCoV-HKU1 and HCoV-NL63) circulate annually in humans and generally cause mild respiratory diseases, although severity can be greater in infants, elderly, and the immunocompromised. In contrast, the Middle East Respiratory Syndrome coronavirus (MERS-CoV) and the Severe Acute Respiratory Syndrome coronavirus (SARS-CoV), belonging to beta-coronavirus lineages C and B, respectively, are highly pathogenic.

In 2019, a novel coronavirus (2019-nCoV/SARS-CoV-2) instigated a major outbreak of respiratory disease Taxonomically, SARS-CoV-2 is a beta-coronavirus, which is thought to be of lineage A or C (Jaimes et al, “Phylogenetic Analysis and Structural Modeling of SARS-CoV-2 Spike Protein Reveals an Evolutionary Distinct and Proteolytically Sensitive Activation Loop,” J. Mol. Biol. (May 1, 2020) 432(10): 3309-3325). COVID-19, the disease caused by SARS-CoV-2, may manifest with a number of clinical symptoms, including pneumonia, fever, dry cough, headache, and dyspnea. In some instances, the disease may progress to respiratory failure and death. Id.

A diagnostic test for determining if a patient has COVID-19 is a real time reverse transcription polymerase chain reaction (RT-PCR) test for the qualitative detection of nucleic acid from SARS-CoV-2 in respiratory samples. The test is used to identify SARS-CoV-2 RNA in a patient sample, and a positive test result indicates the patient has an active coronavirus infection. In a typical protocol, a patient or healthcare provider collects a respiratory sample from the nose or throat of the patient using a swab. The swab is placed in a sealed, sterile container and transported to a laboratory within 72 hours. At the laboratory, viral RNA is extracted from the swab and RT-PCR is performed where viral RNA is reverse transcribed to DNA and then amplified using primers specific to regions of the viral genome. The presence of the DNA may then be indicated with probes that provide a fluorescent signal when bound to the DNA. The RT-PCR test may be administered to individual samples including self-collected nasal swab specimens or with pooled samples.

### SUMMARY

Current strategies for SARS-CoV-2 testing include reverse transcription polymerase chain reaction (RT-PCR), which are time consuming and do not provide immediate results. Of interest would be a fast, reliable SARS-CoV-2 testing device and method which provide accurate, rapid results in a point of care setting.

Lateral flow assay (LFA) devices for detecting whether SARS-CoV-2 nucleocapsid protein is present in a sample are provided. Aspects of the LFA devices include: a sample receiving region; a conjugate region downstream from the sample receiving region that includes test particulate labels made up of label particles conjugated to first and second specific binding members that specifically bind to the SARS-CoV-2 nucleocapsid protein; and a detection region downstream from the conjugate region which includes an immobilized capture specific binding member that specifically binds to the SARS-CoV-2 nucleocapsid protein. Also provided are methods of using the LFA devices, as well as readers, systems and kits for use in the same.

### BRIEF DESCRIPTION OF THE FIGURES

FIGS. 1 to 3 provide various views of an LFA device according to an embodiment of the invention.

FIG. 4 provides a view of the LFA device illustrated in FIGS. 1 to 3 being read with a Veritor™ reader.

### DETAILED DESCRIPTION

Lateral flow assay (LFA) devices for detecting whether SARS-CoV-2 nucleocapsid protein is present in a sample are provided. Aspects of the LFA devices include: a sample receiving region; a conjugate region downstream from the sample receiving region that includes test particulate labels made up of label particles conjugated to first and second specific binding members that specifically bind to the SARS-CoV-2 nucleocapsid protein; and a detection region downstream from the conjugate region which includes an immobilized capture specific binding member that specifically binds to the SARS-CoV-2 nucleocapsid protein. Also provided are methods of using the LFA devices, as well as readers, systems and kits for use in the same.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Lateral Flow Assay Devices

As summarized above, lateral flow assay devices configured for detecting whether SARS-CoV-2 nucleocapsid protein is present in a sample are provided. As used herein the term “lateral flow” refers to liquid flow along the plane of a carrier. As the assay devices are “lateral flow” assay devices, they are configured to receive a sample of interest at a sample receiving region and to provide for the sample to move laterally by capillary action through a conjugate region to a detection region, such that the sample is wicked laterally along the device from the sample receiving region through a conjugate region to the detection region by capillary action. The sample receiving region, conjugate region and detection region may be part of a capillary flow member that is made up of a material that supports capillary flow from the sample region through the conjugate region to the detection region. The capillary flow member may be fabricated from any convenient material. Examples of suitable materials include highly absorbent or bibulous materials, where bibulous materials of interest include, but are not limited to: organic or inorganic polymers, and natural and synthetic polymers. More specific examples of suitable highly absorbent or bibulous materials include, without limitation, glass, glass fiber, cellulose, nylon, crosslinked dextran, untreated paper, porous paper, various chromatographic papers, nitrocellulose, nitrocellulose blends with polyester or cellulose, rayon, acrylonitrile copolymer and plastic. While the capillary flow member and overall configuration of the lateral flow assay device may vary, in certain embodiments the capillary flow member has a strip configuration. Where the highly absorbent or bibulous material is configured as a strip, the capillary flow member has a length that is longer than its width. While any practical configuration may be employed, in some instances the length is longer than the width by 1.5-fold or more, such as 2-fold or more, e.g., 10-fold or more, including 20-fold or more. In some instances, the length of the bibulous member ranges from 0.5 to 20 cm, such as 1.0 to 15 cm, e.g., 2.0 to 10 cm, while the width ranges 0.1 to 5.0 cm, such as 0.5 to 2.5 cm, e.g., 1 to 2 cm. The thickness of the capillary flow member may also vary, ranging in some instances from 0.01 to 0.05 cm, such as 0.1 to 0.4 cm, e.g., 0.1 to 0.25 cm.

As indicated above, the capillary flow member includes a sample receiving region, a conjugate region and a detection region, where these regions are arranged such that liquid sample added to the sample receiving region flows or wicks through the conjugate region to the detection region and in some instances, e.g., as further described below, to further downstream regions, e.g., control regions, wicking regions/absorbent pads, etc. The sample receiving region may simply be a first region of the capillary flow member, e.g., a region positioned closer to one end, which may be viewed as the proximal end, of the capillary flow member. Alternatively, the sample receiving region may be distinct from the capillary flow member but configured to provide for fluid communication of sample into the capillary flow member upon application of sample to the sample receiving region. The sample receiving region may be configured to receive samples of varying volumes, where in some instances the sample receiving region is configured to receive a sample having a volume ranging from 0.1 to 1000 μl, such as 5 to 20 μl and including 50 to 200 μl.

In addition to the sample receiving region, lateral flow assay devices of the invention further include a conjugate region. The conjugate region is a region that includes test particulate labels made up of label particles conjugated to first and second specific binding members that specifically bind to the SARS-CoV-2 nucleocapsid protein. The test particulate labels are non-stably associated with the absorbent material in the conjugate region. By “non-stably associated” is meant that while the test particulate labels may be stationary relative to the absorbent material prior to sample application, upon sample application and sample wicking through the conjugate region, the test particulate labels are free to react with analyte, e.g., SARS-CoV-2 nucleocapsid protein, present in the sample and to move with the sample through the absorbent material of the capillary flow member by capillary action. As such, the test particulate labels move laterally through the absorbent material under the bulk fluid flow forces,

Test particulate labels present in the conjugate region include label particles stably associated with both first and second specific binding members that are distinct from each other (i.e., have different sequences) and specifically bind to the SARS-CoV-2 nucleocapsid protein. As the first and second specific binding members are stably associated with a label particle in a test particulate label, they do not disassociate from the label particle under the assay conditions of the LFA devices of the invention. The stable association of the specific binding members with the label particles may be achieved via covalent or non-covalent binding, as desired.

The label particles of the test particulate labels may vary, as desired. The label particles are optically detectable particles that may be fabricated from a variety of materials, such as metals, e.g., gold, or colored glass or plastic (e.g., polystyrene, polypropylene, latex beads). The label particles may vary in diameter, where label particle diameter in some instances may range from 1 to 5000 nm, such as 1 to 2500 nm. In some instances, the label particles are reflective nanoparticles, e.g., metallic, reflective nanoparticles, such as gold reflective nanoparticles. The term “nanoparticle” as used herein, refers to particles having one dimension in the range of 1 to 1000 nanometers (“nm”). The nanoparticles of the invention may be of any shape. In certain embodiments the nanoparticles are spherical.

As described above, the test particulate labels have stably associated therewith first and second binding members that specifically bind to the SARS-CoV-2 nucleocapsid (i.e., N) protein. The terms “specific binding,” “specifically bind,” and the like, refer to the ability of the binding member to preferentially bind directly to the SARS-CoV-2 nucleocapsid protein relative to other molecules or moieties in a solution or reaction mixture that may be present in the LFA. In certain embodiments, the affinity between the first and second binding members and the SARS-CoV-2 nucleocapsid protein to which they specifically bind when they are specifically bound to each other in a binding complex is characterized by a KD (dissociation constant) of 10⁻⁶ M or less, such as 10⁷ M or less, including 10⁻⁸ M or less, e.g., 10⁻⁸ M or less, including 10⁻¹³ M, such as10⁻¹¹ M or less, e.g., 10⁻¹² M or less, where in some instances the KD is 10⁻¹³ M or less, such as 10⁻¹⁴ M or less, e.g., 10⁻¹⁵ M or less. A variety of different types of specific binding agents may be employed as first and second specific binding members. In some instances, the first and second binding members are antibody binding agents. The term “antibody binding agent” as used herein includes polyclonal or monoclonal antibodies or fragments thereof that are sufficient to specifically bind to the SARS-CoV-2 nucleocapsid (i.e., N) protein. The antibody fragments can be, for example, monomeric Fab fragments, monomeric Fab′ fragments, or dimeric F(ab)′2 fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies. In some instances, the first and second specific binding members are monoclonal antibodies that specifically bind to the SARS-CoV-2 nucleocapsid protein. In some instances, the particles may have three or more specific binding that specifically bind to the SARS-CoV-2 nucleocapsid protein, where in such instances the number of specific binding members that specifically bind to the SARS-CoV-2 nucleocapsid protein may vary, and in some instances may range from three to ten, such as three to five. In some instances, the specific binding members are chosen to bind to different epitopes of the target analyte. The amounts of the various antibodies may vary as desired. In some instances, the amount of given antibody ranges from 2.5-97.5%. In some instances, the amounts of the different antibodies are the same. In yet other embodiments, the amounts of the various antibodies are different.

The SARS-CoV-2 nucleocapsid protein is described in Dutta et la. (2020) Journal of Virology 94(13): e00647-20; Zeng et al. (2020) Biochem Biophys Res Commun. 527(3): 618-623; and Kang et al. (2020) Acta Pharmaceutica Sinica B 10(7):1228-1238, the disclosures of which are incorporated herein by reference in their entireties. In some instances, the first and second binding members may be cross reactive with the SARS-CoV nucleocapsid protein. The SARS-CoV nucleocapsid protein and/or exemplary antigenic determinants of interest on a SARS-CoV nucleocapsid protein are described in U.S. Pat. Nos. 7,696,330; 7,897,744; 7,696,330; 8,343,718; U.S. Publication No.'s: 20080269115; 20100172917; 20090280507; 20080254440; 20070128217, the disclosures of which are incorporated by reference herein in their entireties. In such instances, the first and second binding members may not be cross-reactive with other coronaviral nucleocapsid proteins, e.g., MERS-CoV Nucleoprotein protein; HCoV-229E Nucleoprotein protein; HCoV-NL63 Nucleoprotein protein; HCoV-HKU1(isolate N5) Nucleoprotein protein; and HCoV-OC43 Nucleoprotein.

Where the first and second specific binding members of the test particulate labels are antibody binding agents, examples of antibody binding agents that may be employed include, but are not limited to, those described in U.S. Pat. No. 7,696,330 as well as those described in published United States Patent Application Publication Nos. US20160238601; US20090280507; and US20060003340; the disclosures of which are herein incorporated by reference. In certain embodiments the antibodies are “mammalian”, such that they are obtained from organisms which are within the class mammalia, including the orders carnivore (e.g., dogs and cats), rodentia (e.g., mice, guinea pigs, and rats), and primates (e.g., humans, chimpanzees, and monkeys). In some instances, the antibodies are human, mouse (murine) or rabbit (leporine) antibodies, Specific antibodies of interest that may be employed as first and second binding members of the test particulate labels include, but are not limited to: SARS Nucleocapsid Protein Antibody (Novas); Anti-SARS-CoV-2 Nucleocapsid Antibody, clone 503 (Sigma Aldrich); SARS-CoV-2 (COVID-19) nucleocapsid antibody [HL5511](Genetex)(rabbit monoclonal); SARS-CoV-2 (COVID-19) nucleocapsid antibody [HL455-MS](Genetex)(mouse monoclonal); SARS-CoV-2 (COVID-19) nucleocapsid antibody [HL344](Genetex)(rabbit monoclonal); SARS-CoV-2 (COVID-19) nucleocapsid antibody [HL5410](Genetex)(rabbit monoclonal);SARS-CoV/SARS-CoV-2 Nucleocapsid Monoclonal Antibody (6H3) (Invitrogen) (mouse monoclonal); SARS/SARS-CoV-2 Coronavirus Nucleocapsid Monoclonal Antibody (E16C) (Invitrogen) (mouse monoclonal); SARS/SARS-Cov-2 Coronavirus Nucleocapsid Monoclonal Antibody (5) (Invitrogen) (mouse monoclonal): SARS/SARS-CoV-2 Coronavirus Nucleocapsid Recombinant Rabbit Monoclonal Antibody (1) (Invitrogen); SARS-CoV-2 Nucleocapsid Chimeric Recombinant Human Monoclonal Antibody (1A6) (Invitrogen); SARS Coronavirus Nucleocapsid Monoclonal Antibody (18F629.1) (Invitrogen)(mouse monoclonal); SARS-CoV-2 Nucleocapsid Chimeric Recombinant Human Monoclonal Antibody (1A6)(Invitrogen); SARS-CoV-2 Nucleocapsid Monoclonal Antibody (bcn1)(Invitrogen)(human monoclonal); SARS-CoV-2 Nucleocapsid Monoclonal Antibody (bcn05)(Invitrogen)(human monoclonal); SARS-CoV-2 Nucleocapsid Monoclonal Antibody (bcn14)(Invitrogen) (human monoclonal); SARS-CoV-2 Nucleocapsid Monoclonal Antibody (bcn12)(Invitrogen) (human monoclonal); SARS-CoV-2 Nucleocapsid Monoclonal Antibody (bcn13)(Invitrogen) (human monoclonal); SARS-CoV-2 Nucleocapsid Monoclonal Antibody (ARC2372)(Invitrogen)(rabbit monoclonal); 2019-nCoV Nucleocapsid Antibody (HC2003), SARS-CoV-2 NP Antibody; SARS-CoV/SARS-CoV-2 Nucleocapsid Antibody, Rabbit MAb R004 (Sino Biological); SARS-CoV/SARS-CoV-2 Nucleocapsid Antibody, Mouse MAb MM05 (Sino Biological); SARS-CoV/SARS-CoV-2 Nucleocapsid Antibody, Mouse MAb MM08 (Sino Biological); and the like.

In some instances, the first and second specific binding members of the test particulate labels that specifically bind to the SARS-CoV-2 nucleocapsid protein are leporine (rabbit) and murine (mouse) antibodies, respectively. The amounts of the leporine and murine antibodies may vary as desired. In some instances, the amount of leporine antibody ranges from 2.5-97.5% and the amount of the murine antibody ranges from 2.5-97.5%. In some instances, the amounts of the leporine and murine antibodies are the same. In yet other embodiments, the amounts of the leporine and murine antibodies are different. Specific examples of both leporine and murine monoclonal antibodies that specifically bind to SARS-CoV-2 Nucleocapsid protein are provided above. In some such embodiments, the leporine antibody is R004 and the murine antibody is MM05 (Sino Biological). In some such embodiments, the amount of leporine antibody exceeds the amount of murine antibody, where in some instances the percentage of the first, leporine, antibody ranges from over 50% to 97.5%, such as 60 to 97%, e.g., 75 to 95%, such as 80 to 90%, e.g., 85%.

As summarized above, downstream from the conjugate region is a detection region. A detection region is a region of the capillary flow member from which a result may be read during use of the device. The detection region is positioned at some distance downstream from the sample receiving region of the device. By “downstream” is meant the lateral direction that the sample flows by capillary action, i.e., the direction of fluid flow from the sample receiving region. The distance between the sample receiving region and the detection region may vary, ranging in some instances from 0.3 to 15 cm, such as 1 to 15 cm and including 5 to 10 cm, e.g., 1 to 5 cm.

The detection region is a region that includes an immobilized capture specific binding member that specifically binds to the SARS-CoV-2 nucleocapsid protein. The detection region includes an amount of capture specific binding member stably associated with the absorbent material of the capillary flow member in the detection region. The size of the detection region may vary, and in some instances the detection region has an area ranging from 0.01 to 0.5 cm², such as 0.05 to 0.1 cm² and including 0.1 to 0.2 cm². The detection region may have a variety of different configurations, where the configuration may be a line, circle, square, or more complex shape, such as a “+”, as desired. In some instances, the detection region is configured as a line of immobilized capture specific binding member, where the dimensions of the line may vary, where in some instances the line ranges in length from 2 to 10 mm, such as 3 to 7 mm, e.g., 4 to 6 mm. As indicated above, the detection region includes a capture specific binding member stably associated with the absorbent material of the capillary flow member. By “stably associated with” is meant that the capture specific binding member and the absorbent material maintain their position relative to each other in space under the conditions of use, e.g., under the assay conditions. As such, the capture specific binding member and the absorbent material of the capillary flow member can be non-covalently or covalently stably associated with each other. Examples of non-covalent association include non-specific adsorption, binding based on electrostatic (e.g., ion-ion pair interactions), hydrophobic interactions, hydrogen bonding interactions, and the like. Examples of covalent binding include covalent bonds formed between the capture specific binding member and a functional group present on the absorbent material. The immobilized capture specific binding member of the detection region that specifically binds to the SARS-CoV-2 nucleocapsid protein is a distinct specific binding member that differs from the first and second specific binding members of the test particulate labels, described above. The immobilized capture specific binding member of the detection region that specifically binds to the SARS-CoV-2 nucleocapsid protein may vary, where examples of such specific binding members are described above. In embodiments, the capture specific binding member is a specific binding member that can bind to the SARS-CoV-2 nucleocapsid protein at the same time as the first/second specific binding members of the test particulate labels, such that a sandwich of the capture specific binding member, SARS-CoV-2 nucleocapsid protein, and test particulate label may be produced when the SARS-CoV-2 nucleocapsid protein is present in the sample being assayed. In some instances, the immobilized capture specific binding member of the detection region that specifically binds to the SARS-CoV-2 nucleocapsid protein is SARS-CoV/SARS-CoV-2 Nucleocapsid Antibody, Mouse MAb MM08 (Sino Biological).

In some instances, the lateral flow assay device may further include a control region. The control region is located downstream from the detection region. The control region contains immobilized control agents. The immobilized control agents bind specifically to mobile control binding agents to form a control binding pair. Control binding pairs of interest act as internal controls, that is, the control against which the analyte measurement results may be compared on the individual test strip. In some instances, the control region may be described as including a control antigen and the the LFA device may include, e.g., in the sample receiving region and/or conjugate region, mobile control particulate labels that include label particles, which may be the same as the label particles of the test particulate labels, conjugated to a control specific binding member that specifically binds to the control antigen. Although, in general, any convenient control antigen/control specific binding member pairs can be used, in some instances control antigens that do not exist in the sample or do not immunologically cross-react with compounds that exist in the sample are employed. Examples of suitable control binding pairs of interest include, but are not limited to: biotin/anti-biotin IgG; chicken IgY/anti-chicken IgY, etc. In yet other embodiments, the control region may include a control binding member that binds to the first and second specific binding members of the test label particulates, e.g., to the Fc region of the first and second specific binding members.

Optionally, the lateral flow assay device may include a wicking region, e.g., in the form of an absorbent pad, downstream from the detection region and any control region, e.g., at the end distal from the sample receiving region, where the absorbent pad is configured to absorb fluid and reagents present therein that have flowed through the capillary flow member. Where desired, the component parts of the lateral flow assay device may be present in a suitable housing. The housing may be configured to enclose the capillary flow member and other assay components. The housing may be fabricated from any suitable material, where the material may be a material that is sufficiently rigid to maintain the integrity of the bibulous member and other components housed therein and also inert to the various fluids and reagents that contact the housing during use. Housing materials of interest include plastics. The housing may include a port or analogous structure configured to allow sample application to the sample application region and a window configured to allow viewing of the detection region. The housing may further include markings, e.g., detection region and control region markings (e.g., “T” and “C”), etc. The housing may comprise a barcode on the outside that may convey information to the tester when scanned by a barcode reader. For example, the barcode may identify the type of test being run and/or the individual lateral flow assay device.

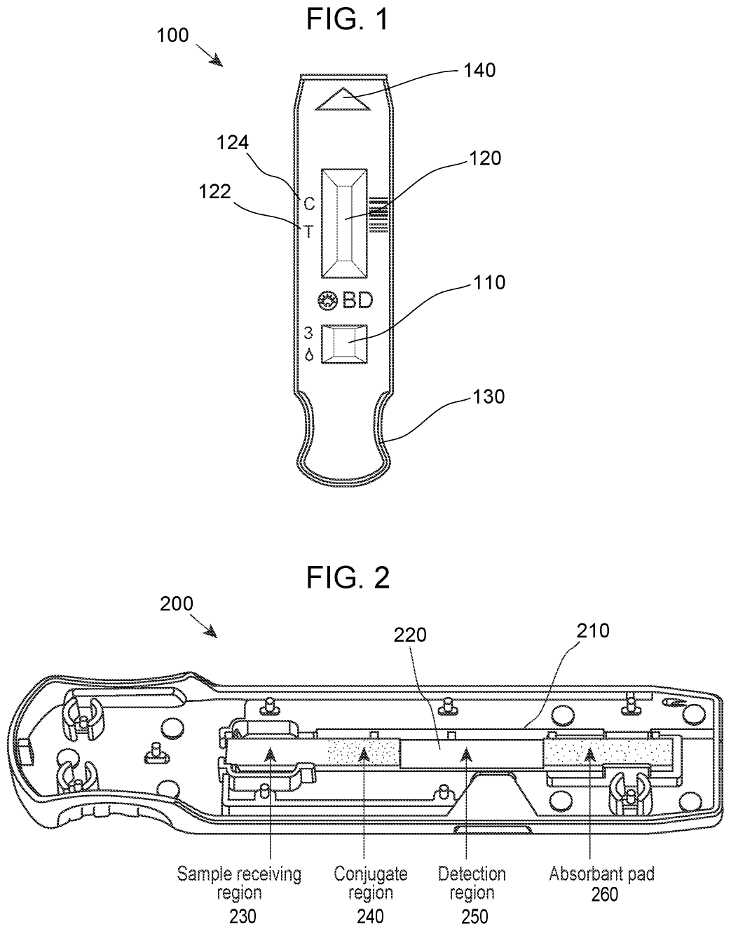

FIG. 1 provides an overhead view of a device according to any embodiment of the invention. In FIG. 1, device 100 includes a port 110 for receiving a sample and a window 120 for viewing the of the detection region. Also shown are markers 122 and 124 for the test and control lines of the detection region viewable via window 120, respectively. In addition, the device includes a handle 130 for use in manipulating the device at a first end and an arrow 140 at the opposite indication to provide guidance for use with an analyzer instrument. FIG. 2 provides a view of a base 200 of a device shown in FIG. 1. In FIG. 2, base 200 including a central region 210 for holding a lateral flow assay test strip 220. Lateral flow assay test strip 220 includes a sample receiving region 230, a conjugate region 240, a detection region 250 and an absorbent pad 260. FIG. 3 provides a perspective view of the device shown in FIGS. 1 and 2.

Devices of the invention may be configured to assay for one or more additional analytes, in addition to the SARS-CoV-2 nucleocapsid protein. Additional analytes for which the device may be configured to assay include, but are not limited to: biological or environmental substances of interest, e.g., viral antigens, such as influenza virus antigens, e.g., influenza A virus, influenza B virus, or influenza C virus, and combinations thereof. Further details regarding detection of such analytes in a lateral flow device are provided in PCT published application WO2019245744; the disclosure of which is herein incorporated by reference.

### Methods

As summarized above, aspects of the invention also include methods of using lateral flow assay devices of the invention, e.g., as described above, to detect whether a SARS-CoV-2 nucleocapsid protein is present in a sample. As such, methods of determining whether a given sample includes or does not include SARS-CoV-2 nucleocapsid protein are provided. In other words, methods of determining that a sample does or does not include SARS-CoV-2 nucleocapsid protein are provided. By “determining” is meant assaying a sample for a signal associated with a component, e.g., nucleocapsid protein, in the sample, wherein the presence of the signal indicates that the component is present in the sample. The determining may include obtaining the signal by visual or instrumental means. In some cases, the determining includes detecting a signal from a sample, e.g., from a component in the sample, where the signal indicates the component is present in the sample.

In practicing methods of the invention, a sample of interest is applied to the sample receiving region of a lateral flow assay device, such as described above. In some instances, the sample is combined with an amount of test particulate labels and/or control particulate labels, e.g., where either or both of these components are not already present in the device, such as described above. When the sample is combined with either or both of these assay components, the combination may be achieved using any convenient protocol. The amount of these agents, when combined with the sample, may vary, with the desired amount being readily determined, e.g., via standard methods known in the art. In such instances, a given LFA device may not include a conjugate region, e.g., as described above.

The amount of sample that is applied to the sample receiving region may vary, so long as it is sufficient to provide for the desired lateral flow and operability of the assay. The sample may be applied to the sample receiving region using any convenient protocol, e.g., via dropper, pipette, syringe and the like. In some instances, the sample is applied directly from a sample obtainment device, such as liquid container, used in obtainment of the sample, e.g., as described below. As such, an initial step in methods of the invention is applying the sample to a sample receiving region of a lateral flow assay device configured to detect SARS-CoV-2 nucleocapsid protein in the sample. In addition to applying sample, the methods may further include applying a quantity of a suitable liquid, e.g., buffer, to provide for adequate fluid flow through the capillary flow member. Any suitable liquid may be employed, including but not limited to buffers, cell culture media (e.g., DNEM), etc. Buffers of interest include, but not limited to: tris, tricine, MOPS, HEPES, PIPES, MES, PBS, TBS, and the like. Where desired, detergents may be present in the liquid, e.g., NP-40 or TWEEN™ detergents. In some embodiments a biological sample is added to a sample buffer liquid or an extraction buffer liquid and mixed, and the resulting mixture is applied to the sample receiving region of a lateral flow assay device.

Following sample application, the sample is allowed to laterally flow through the capillary flow member and various regions thereof, e.g., conjugate region and detection region, and the detection region is then read to determine whether SARS-CoV-2 nucleocapsid protein is present in the non-diagnostic sample. The detection region may be read after a predetermined period of time following sample application, where this period of time may range from 10 sec to 1 hour, such as 1 min to 45 min, e.g., 5 min to 30 min, including 10 min to 20 min, e.g., 15 min.

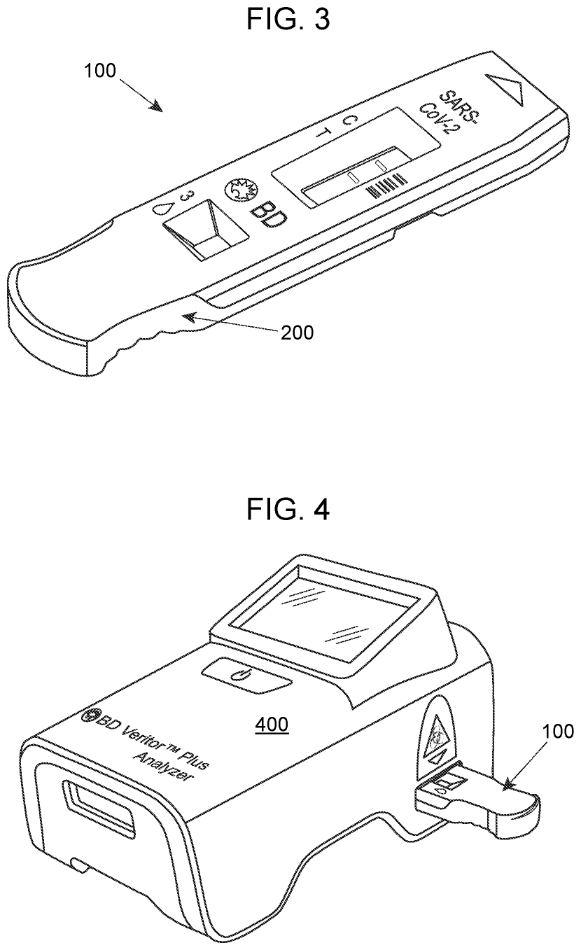

The detection region is read using a protocol that is configured to detect the label particles of the test particulate labels. In some embodiments, a color change can be measured using a reflectance reader. In some embodiments, a reflectance reader refers to an instrument adapted to read a test strip using reflected light, including fluorescence, or electromagnetic radiation of any wavelength. Reflectance can be detected using a photodetector or other detector, such as charge coupled diodes (CCD). In some embodiments, the reader includes the reader of the Veritor™ System (Becton, Dickinson and Company). An illustration of a device 100 as illustrated in FIGS. 1 to 3 being read with the Veritor™ System 400 is shown in FIG. 4. In some embodiments, the reader includes the Sofia or Sofia2 Fluorescent Immunoassay Analyzer (Quidel), the LumiraDx Instrument for reading fluorescence from LurniraDx Test Strips (LumiraDx), and the Alere Reader for reading BinaxNow antigen cards (Abbott). As described above, LFA devices of the invention may include a control region. In such instances, methods of the invention further include reading the control region to obtain a signal therefrom, e.g., with the reflectance reader employed to read the detection region.

Where desired, methods may further include applying a control sample, e.g., positive or negative control, to a sample receiving region of a control lateral flow assay device and reading a detection region of the control lateral flow assay device to obtain a result. In these embodiments, the control lateral flow assay device is identical (e.g., a second lateral flow device from the same production lot as the test lateral flow device) to the test lateral flow assay device. A positive control sample is a fluid sample known to contain a detectable amount of the SARS-CoV-2 nucleocapsid protein. A negative control sample is a fluid sample that is known not to contain a detectable amount of the SARS-CoV-2 nucleocapsid protein. As such, these embodiments employ running a complete positive and/or negative control assay using a lateral flow assay device(s) that is the same as the test lateral flow assay device.

Methods of the invention may provide qualitative or quantitative results. Qualitative results include results that provide a simple “yes” or “no” determination of whether the analyte is present in the sample being assayed. Qualitative results also include results that are positive if the amount of analyte in the sample exceeds a pre-determined threshold. In contrast, quantitative results provide some measurement of how much of the SARS-CoV-2 nucleocapsid protein is present in the sample being assayed. Accordingly, a quantitative result provides at least an approximation of the amount of the SARS-CoV-2 nucleocapsid protein that is present in the sample being assayed. To provide for quantitative results, the detection region may include two or more distinct capture probe regions that include the same or different amounts of the same capture probe. As such, if the amount of analyte in the sample exceeds the amount of the analyte that can be captured in the first capture region, the remaining free analyte will move to the second capture region. The resultant positive results from the both regions provide a quantitative measurement of the amount of analyte in the sample. By having a series of regions, which may be a gradient of two or more capture regions each having differing (such as decreasing) amounts of capture probe, a quantitative measurement of the analyte in the sample may be obtained. Alternatively, quantitative measurements can be obtained by densitometry. In this case, only one capture region is necessary.

The sample that is assayed in accordance with embodiments of the invention may vary. Examples of samples may include various fluid or solid samples. In some instances, the sample can be a bodily fluid sample from a subject. The sample can be an aqueous or gaseous sample. In some instances, solid or semi-solid samples can be provided. The sample can include tissues and/or cells collected from the subject. The sample can be a biological sample. Examples of biological samples can include but are not limited to, blood, serum, plasma, nasal swab or nasopharyngeal wash, saliva, urine, gastric fluid, spinal fluid, tears, stool, mucus, sweat, earwax, oil, glandular secretion, cerebral spinal fluid, tissue, semen, vaginal fluid, interstitial fluids derived from tumorous tissue, ocular fluids, spinal fluid, throat swab, breath, hair, finger nails, skin, biopsy, placental fluid, amniotic fluid, cord blood, emphatic fluids, cavity fluids, sputum, pus, micropiota, meconium, breast milk and/or other excretions. The samples may include nasopharyngeal wash. Examples of tissue samples of the subject may include but are not limited to, connective tissue, muscle tissue, nervous tissue, epithelial tissue, cartilage, cancerous sample, or bone. The sample may be provided from a human or animal. The sample may be provided from a mammal, vertebrate, such as murines, simians, humans, farm animals, sport animals, or pets. The sample may be collected from a living or dead subject. The sample may be collected fresh from a subject or may have undergone some form of pre-processing, storage, or transport. In certain embodiments the source of the sample is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some instances, the subjects are humans. The methods may be applied to samples obtained from human subjects of both genders and at any stage of development (i.e., neonates, infant, juvenile, adolescent, adult), where in certain embodiments the human subject is a juvenile, adolescent or adult.

Methods of the invention may include obtaining a sample from a subject. For example, where the sample to be tested is a nasopharyngeal sample or specimen, e.g., nasal swab, the methods may include obtaining the sample from a subject using a swab, nasopharyngeal wash, etc. In some instances, obtaining a sample from a subject includes obtaining a nasal swab specimen from the subject using the dual nares collection method. In embodiments of this method, a nasal swab is first inserted into one nostril of a subject. The swab tip is inserted up to 2.5 cm (1 inch) from the edge of the nostril. The swab is rolled 5 times along the mucosa inside the nostril to ensure that both mucus and cells are collected. The same swab is then used to repeat this process for the other nostril to ensure that an adequate sample is collected from both nasal cavities. The swab is then removed from the nasal cavity.

Following obtainment of the sample, such as nasal swab, from the subject, the sample may be processed, as desired, prior to application to the sample receiving region of the LFA device. For example, the sample may be combined with detergent, preservative, etc., in an aqueous vehicle to prepare the sample for testing. In some instances, the sample is collected using the Veritor™ (Becton, Dickinson and Company) sample collection system. In such instances, a cap is first removed from a Veritor™ extraction reagent tube/tip and then the swab with the collected nasopharyngeal specimen is inserted into the tube, followed by plunging the swab up and down in the fluid provided in the tube for a minimum of 15 seconds, taking care not to splash contents out of the tube. The swab is then removed from the tube while squeezing the sides of the tube to extract the liquid from the swab. The attached tip is then firmly pressed onto the extraction reagent tube containing the processed sample (threading or twisting is not required). The contents are then mixed thoroughly by swirling or flicking the bottom of the tube.

Embodiments of the invention provide for fast, reliable determination of whether a given sample contains the SARS-CoV-2 nucleocapsid protein. Results can be obtained in embodiments of the invention within 30 minutes, such as within 20 minutes, including within 15 minutes, of applying a sample to a sample receiving region of an LFA device. Embodiments of the methods provide: a PPA (Positive Percent Agreement=True Positives/True Positives+False Negatives) of 70% or greater, such as 75% or greater, including 80% or greater, e.g., 84% or greater; an NPA (Negative Percent Agreement True Negatives/True Negatives+False Positives) of 90% or greater, such as 95% or greater, including 100%; and an OPA (Overall Percent Agreement=True Positives+True Negatives/Total Samples) of 90% or greater, such as 95% or greater, including 98% or greater. Embodiments of the methods provide an Limit of Detection (LOD) of 1.4×10² TCID₅₀/mL or less, Embodiments of the methods show no cross-reactivity with a variety of potential cross contaminating entities, including but not limited to: Human coronavirus 229E (heat inactivated); Human coronavirus OC43; Human coronavirus NL63; Adenovirus; Human Metapneumovirus; Parainfluenza virus 1; Parainfluenza virus 2; Parainfluenza virus 3 Parainfluenza virus 4; Influenza A; Influenza B; Enterovirus; Respiratory syncytial virus; Rhinovirus; SARS-coronavirus; MERS-coronavirus; Haemophilus influenza; Streptococcus pneumoniae; Streptococcus pyogenes; Candida albicans; Pooled human nasal wash; Bordetella pertussis; Mycoplasma pneumoniae; Chlamydia pneumoniae; and Legionella pneumophila.

Methods of embodiments of the invention may include assaying for one or more additional analytes, in addition to the SARS-CoV-2 nucleocapsid protein. Additional analytes which may be assayed in accordance with embodiments of the invention include, but are not limited to: biological or environmental substances of interest, e.g., viral antigens, such as influenza virus antigens, e.g., influenza A virus, influenza B virus, or influenza C virus, and combinations thereof. Further details regarding detection of such analytes in a lateral flow device are provided in POT published application WO2019245744; the disclosure of which is herein incorporated by reference.

### Utility

The subject devices and methods find use in clinical and research applications where detection of SARS-CoV-2 nucleocapsid protein in a sample is desired. Embodiments of the invention provide for fast, reliable SARS-CoV-2 testing. Embodiments of the invention provide for lab-quality results at the point of care, in a simple-to-operate, handheld instrument.

### Kits

Aspects of the present disclosure also include kits. The kits may be suitable for practicing any of the subject methods. Kits may include one or more, including a plurality of, e.g., 2 to 50, such as 5 to 30, lateral flow assay devices, e.g., as described above. The kits may further include one or more additional assay components, such as but not limited to, sample obtainment devices, e.g., nasal swabs/liquid containers (e.g., in the form of extraction tubes (where the tubes may include a reagent liquid, such as an aqueous liquid comprising a detergent, preservative, etc.)), a positive control, e.g., in the form of a positive control swab that includes the SARS-CoV-2 nucleocapsid protein, a negative control, e.g., in the form of a negative control swab that does not include the SARS-CoV-2 nucleocapsid protein, etc. In some instances, the kits of the invention include 2 to 50, such as 5 to 30, lateral flow assay devices, 2 to 50, such as 5 to 30 sample obtainment components (e.g., in the form of a nasal swab/extraction tube), a positive control swab and a negative control swab. The various components of the kits may be present in separate containers, or some or all of them may be pre-combined into the same containers. The containers may be configured to preserve the sterility of the components, e.g., foil pouches, etc. As such, the kit components may be sterile and present in one or more sterile containers, such as foil pouches. The kit components may be combined in any convenient form of packaging, e.g., in a box, pouch or other type of packaging, as desired.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., diskette, compact disk (CD), etc., portable flash drive, etc., on which the information has been recorded. Yet another form of these instructions that may be present is a website address which may be used via the Internet to access the information at a removed site.

The following example(s) is/are offered by way of illustration and not by way of limitation.

### EXAMPLES

### I. Comparison of Colloidal Gold Conjugates

A test colloidal gold conjugate was made with 85% anti-SARS-CoV-2 antibody R004 (Sino Biological) and 15% anti-SARS-CoV-2 antibody MMOS (Sino Biological), and a control colloidal gold conjugate was made using 100% anti-SARS-CoV-2 antibody R004. Two lateral flow assays (LFAs), i.e., a test LEA and a control LEA, were made using the same striped nitrocellulose membrane and the test and control colloidal gold conjugates, respectively, SARS-CoV-2 negative nasal fluid and nasal swab clinical samples were extracted in an Extraction Reagent and the extracted samples were applied to the test and control LFAs. The results showed that the test LEA assay with the test colloidal gold conjugate containing two different antibodies, i.e., R004 and MM05, had better specificity, less false positive results, as compared to the control LFA assay with the control colloidal gold conjugate containing only one antibody, i.e., R004.

### II. Veritor™ System for Rapid Detection of SARS-CoV-2

### A. Summary

The BD Veritor™ System for Rapid Detection of SARS-CoV-2 is a rapid (approximately 15 minutes) chromatographic digital immunoassay for the direct detection of the presence or absence SARS-CoV-2 antigens in respiratory specimens taken from patients with signs and symptoms who are suspected of COVD-19.

### B. Principles of the Procedure

The BD Veritor™ System employs a dedicated opto-electronic interpretation instrument and immunochromatographic assays for the qualitative detection of antigens from pathogenic organisms in samples processed from respiratory specimens. The BD Veritor™ System for Rapid Detection of SARS-CoV-2 is designed to detect the presence or absence of SARS-CoV-2 nucleocapsid proteins in respiratory samples from patients with signs and symptoms of infection who are suspected of COVID-19. When specimens are processed and added to the test device, SARS-CoV-2 antigens present in the specimen bind to antibodies conjugated to detector particles in the test strip. The antigen-conjugate complexes migrate across the test strip to the reaction area and are captured by a line of antibodies bound on the membrane. A positive result is determined by the BD Veritor™ Plus Analyzer when antigen-conjugate is deposited at the Test “T” position and the Control “C” position on the assay device. The instrument analyzes and corrects for non-specific binding and detects positives not recognized by the unaided eye to provide an objective result.

### C. Materials

### 1. Kit Components

The following components are included in the BD Veritor System for Rapid Detection of SARS-CoV-2 kit.

| Kit Component | Quantity | Description |
|---|---|---|
| BD Veritor System | 30 single use test devices | Foil pouched test device |
| Test Devices | | containing one reactive strip. |
| | | Each strip has one line of murine |
| | | anti-SARS coronavirus |
| | | monoclonal antibody on the test |
| | | line, and one of biotin coupled to |
| | | bovine protein on the positive |
| | | control line. |
| | | Murine and Leporine anti-SARS |
| | | coronavirus and |
| | | anti-biotin monoclonal antibodies |
| | | conjugated to detector reagents |
| | | are bound in the sample delivery |
| | | area. |
| Extraction | 30 single use reaction tubes, | Detergent solution with less |
| Reagent | each with 325 μL extraction | than 0.1% sodium azide |
| | reagent and having an | (preservative). |
| | integral dispensing tip | |
| Specimen | 30 sterile, single use | For sample collection and |
| sampling swabs | specimen sampling swabs | transfer. |
| SARS-CoV-2 (+) | 1 each - individually wrapped | Non-infectious, recombinant |
| Control Swab | for single use | viral protein antigen with less |
| | | than 0.1% sodium azide. |
| SARS-CoV-2 (−) | 1 each - individually wrapped | Buffer with less than 0.1% |
| Control Swab | for single use | sodium azide. |
| Assay | 1 each - Instructions for use | |
| documentation | 1 each - Quick reference | |
| | instruction card 1 each - Nasal | |
| | sampling instructions | |

### 2. Analyzer

BD Veritor™ Plus Analyzer (Cat. No. 256066)

### C. Conical Performance

The performance of the BD Veritor™ System for Rapid Detection of SARS-CoV-2 was established with 226 direct nasal swabs prospectively collected and enrolled from individual symptomatic patients (within 5 days of onset) who were suspected of COVID-19. Samples were collected by qualified personnel in 21 geographically diverse areas across the United States.

Nasal swabs were collected following the dual nares method and handled as described in the package insert of the collection device. Specimens were frozen within 30 minutes of collection and stored until tested. All specimens within a prespecified date range were selected and then sequentially tested in a blinded fashion. The performance of the BD Veritor™ System Assay was compared to results of a nasopharyngeal or oropharyngeal swab stored in 3 mL viral transport media tested with an Emergency Use Authorized molecular (RT-PCR) test for detection of SARS-CoV-2. The results are provided in Table 1, below.

TABLE 1

| BD Veritor | Reference PCR Results | | |
|---|---|---|---|
| Results | POS | NEG | Total |
| POS | 26 | 0 | 26 |
| NEG | 5 | 195 | 200 |
| Total | 31 | 195 | 226 |

PPA: 84% (C.I. 67%-93%)
NPA: 100% (C.I. 98%-100%)
OPA: 98% (C.I. 95%-99%)
EXPLANATION OF TERMS:
PPA: Positive Percent Agreement = True Positives/True Positives + False Negatives
NPA: Negative Percent Agreement = True Negatives/True Negatives + False Positives.
OPA: Overall Percent Agreement = True Positives + True Negatives/Total Samples
C.I.: Confidence Interval

### D. Limit of Detection (LOD) (Analytical Sensitivity)

The LOD for the BD Veritor™ System for Rapid Detection of SARS-CoV-2 was established using limiting dilutions of a viral sample inactivated by gamma irradiation. The material was supplied at a concentration of 2.8×10⁵ TCID₅₀/mL. In this study, designed to estimate the LOD of the assay when using a direct nasal swab, the starting material was spiked into a volume of pooled human nasal matrix obtained from healthy donors and confirmed negative for SARS-CoV-2. An initial range finding study was performed testing devices in triplicate using a 10-fold dilution series. At each dilution, 50 μL samples were added to swabs and then tested in the BD Veritor™ assay using the procedure appropriate for patient nasal swab specimens. A concentration was chosen between the last dilution to give 3 positive results and the first to give 3 negative results. Using this concentration, the LOD was further refined with a 2-fold dilution series. The last dilution demonstrating 100% positivity was then tested in an additional 20 replicates tested in the same way. The results are provided in Table 2, below

TABLE 2

| Starting Material | Estimated | No. | % |
|---|---|---|---|
| Concentration | LOD | Positive/Total | Positive |
| 2.8 × 10⁵ TCID₅₀/mL | 1.4 × 10² TCID₅₀/mL | 19/20 | 95% |

### E. Cross Reactivity (Analytical Specificity)

Cross-reactivity of the BD Veritor™ System for Rapid Detection of SARS-CoV-2 was evaluated by testing a panel of high prevalence respiratory pathogens that could potentially cross-react with the BD Veritor™ System for Rapid Detection of SARS-CoV-2. Each organism/virus pair was tested in triplicate. The final concentration of each organism is documented in the following Table 3.

TABLE 3

| Potential Cross- | Concentration | | Cross-Reactivity |
|---|---|---|---|
| Reactant | Tested | | (Yes/No) |
| Human coronavirus 229E | 1.0 × 10⁵ | U/mL | No |
| (heat inactivated) | | | |
| Human coronavirus OC43 | 1.0 × 10⁵ | TCID₅₀/mL | No |
| Human coronavirus NL63 | 1.0 × 10⁵ | TCID₅₀/mL | No |
| Adenovirus | 1.0 × 10⁵ | TCID₅₀/mL | No |
| Human Metapneumovirus | 1.0 × 10⁵ | TCID₅₀/mL | No |
| Parainfluenza virus 1 | 1.0 × 10⁵ | TCID₅₀/mL | No |
| Parainfluenza virus 2 | 1.0 × 10⁵ | TCID₅₀/mL | No |
| Parainfluenza virus 3 | 5.2 × 10⁵ | TCID₅₀/mL | No |
| Parainfluenza virus 4 | 1.6 × 10⁴ | TCID₅₀/mL | No |
| Influenza A | 2.5 × 10⁵ | TCID₅₀/mL | No |
| Influenza B | 2.9 × 105 | TCID50/mL | No |
| Enterovirus | 4.0 × 105 | TCID50/mL | No |
| Respiratory syncytial virus | 4.0 × 105 | TCID50/mL | No |
| Rhinovirus | 1.1 × 105 | PFU/mL | No |
| SARS-coronavirus | 4.5 × 105 | PFU/mL | No |
| MERS-coronavirus | 1.5 × 105 | TCID50/mL | No |
| Haemophilus influenza | 1.4 × 106 | CFU/mL | No |
| Streptococcus pneumoniae | 1.0 × 106 | CFU/mL | No |
| Streptococcus pyogenes | 1.6 × 106 | CFU/mL | No |
| Candida albicans | 1.8 × 106 | CFU/mL | No |
| Pooled human nasal wash | 100% | | No |
| Bordetella pertussis | 1.4 × 106 | CFU/mL | No |
| Mycoplasma pneumoniae | 1.0 × 106 | CFU/mL | No |
| Chlamydia pneumoniae | 1.0 × 106 | IFU/mL | No |
| Legionella pneumophila | 1.0 × 106 | CFU/mL | No |

To estimate the likelihood of cross-reactivity with SARS-CoV-2 of organisms that were not available for wet testing. In silica analysis using the Basic Local Alignment Search Tool (BLAST) managed by the National Center for Biotechnology Information (NCBI) was used to assess the degree of protein sequence homology.
For P. jirovecii one area of sequence similarity shows 45.4% homology across 9% of the sequence, making cross-reactivity in the BD Veritor™ sandwich immunoassay highly unlikely.
No protein sequence homology was found between SARS-CoV-2 and M. tuberculosis, and thus homology-based cross-reactivity can be ruled out.

The comparison between SARS-CoV-2 nucleocapsid protein and human coronavirus HKU1 revealed that the only potential for homology is with the HKU1 nucleocapsid phosphoprotein. Homology is relatively low, at 36.7% across 82% of sequences, but cross-reactivity cannot be ruled out.

### F. High Dose Hook Effect

No high dose hook effect was observed up to 2.8×10⁵ TCID₅₀/mL of gamma-inactivated SARS-CoV-2 with the BD Veritor™ System for Rapid Detection of SARS-CoV-2 test.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A lateral flow assay (LFA) device for detecting whether SARS-CoV-2 nucleocapsid protein is present in a sample, the LFA device comprising:
(a) a sample receiving region;
(b) a conjugate region downstream from the sample receiving region and comprising test particulate labels comprising label particles conjugated to first and second specific binding members that specifically bind to the SARS-CoV-2 nucleocapsid protein; and
(c) a detection region downstream from the conjugate region and comprising an immobilized capture specific binding member that specifically binds to the SARS-CoV-2 nucleocapsid protein.

2. The LFA device according to claim 1, further comprising a control region downstream from the detection region.

3. The LFA device according to claim 2, wherein the control region comprises a control antigen and the device further comprises control particulate labels comprising label particles conjugated to a control specific binding member that specifically binds to the control antigen.

4. The LFA device according to claim 2, wherein the control region comprises a control binding member that binds to the first and second specific binding members.

5. The LFA device according to claim 1, further comprising a wicking region downstream from the control region.

6. The LFA device according to claim 1, wherein the label particles are reflective nanoparticles.

7. The LFA device according to claim 6, wherein the reflective nanoparticles comprise a metal.

8. The LFA device according to claim 7, wherein the metal comprises gold.

9. The LFA device according to claim 1, wherein the first, second and capture specific binding members are antibodies or binding fragments thereof.

10. The LFA device according to claim 9, wherein the first and second specific binding members that specifically bind to the SARS-CoV-2 nucleocapsid protein are leporine and murine antibodies, respectively.

11. The LFA device according to claim 10, wherein the leporine antibody is R004 and the murine antibody is MM05.

12. The LFA device according to claim 11, wherein the leporine antibody is present in an amount that exceeds the murine antibody amount.

13. The LFA device according to claim 12, wherein the amount of leporine antibody ranges from 60 to 97.5%.

14. The LFA device according to claim 1, wherein the capture specific binding member that specifically binds to the SARS-CoV-2 nucleocapsid protein is a murine antibody.

15. The LFA device according to claim 13, wherein the murine antibody is MM08.

16-23. (canceled)

24. A method of detecting whether a SARS-CoV-2 nucleocapsid protein is present in a sample, the method comprising:
(a) placing the sample onto a sample receiving region of a lateral flow assay (LFA) device comprising:
(i) the sample receiving region;
(ii) a conjugate region downstream from the sample receiving region and comprising particulate labels comprising label particles conjugated to first and second specific binding members that specifically bind to the SARS-CoV-2 nucleocapsid protein; and
(iii) a detection region downstream from the conjugate region and comprising an immobilized capture specific binding member that specifically binds to the SARS-CoV-2 nucleocapsid protein; and
(b) interrogating the detection region for the presence of label particles to detect whether the SARS-CoV-2 nucleocapsid protein is present in the sample.

25. The method according to claim 24, wherein the LFA device further comprises a control region downstream from the detection region and the method further comprises interrogating the control region.

26-31. (canceled)

32. The method according to any of claim 24, wherein the first, second and capture specific binding members are antibodies or binding fragments thereof.

33. The method according to claim 32, wherein the first and second specific binding members that specifically bind to the SARS-CoV-2 nucleocapsid protein are leporine and murine antibodies, respectively.

34-35. (canceled)

36. The method according to claim 24, wherein the capture specific binding member that specifically binds to the SARS-CoV-2 nucleocapsid protein is a murine antibody.

37-92. (canceled)
